(19) United States
(12) Patent Application Publication
(10) Pub. No.: US 2014/0005415 A1
(43) Pub. Date: Jan. 2, 2014

(54) IMMOBILIZATION OF IONIC LIQUIDS VIA MECHNOCHEMICAL INTERCALATION IN LAYERED MATERIALS

(71) Applicants: Luyi Sun, Pearland, TX (US); Yuezhong Meng, Guangzhou (CN); Min Xiao, Guangzhou (CN); Hang Hu, Guangzhou (CN); Jarrett Clay Martin, Georgtown, TX (US)

(72) Inventors: Luyi Sun, Pearland, TX (US); Yuezhong Meng, Guangzhou (CN); Min Xiao, Guangzhou (CN); Hang Hu, Guangzhou (CN); Jarrett Clay Martin, Georgtown, TX (US)

(73) Assignee: TEXAS STATE UNIVERSITY-SAN MARCOS, San Marcos, TX (US)

(21) Appl. No.: 13/992,499

(22) PCT Filed: Dec. 7, 2011
(86) PCT No.: PCT/US2011/063783

§ 371 Date: Sep. 20, 2013

Related U.S. Application Data
(60) Provisional application No. 61/459,078, filed on Dec. 7, 2010.

(51) Int. Cl. B01J 31/38; C07D 317/36; B01J 31/26

(52) U.S. Cl. CPC B01J 31/38; B01J 31/26; C07D 317/36
USPC 549/230; 502/150; 502/62

(57) ABSTRACT

A facile mechanochemical intercalation approach was adopted to immobilize ionic liquids into layered materials. The immobilized ionic liquids were found to be useful as catalysts for the coupling reaction of CO2 and propylene oxide to synthesize propylene carbonate. The immobilized ionic liquid exhibited similar reactivity as the free ionic liquid. Overall, the 10 mechanochemical approach proves to be effective in immobilizing ionic liquids in layered compounds and thus may expand the applications of ionic liquids and, meanwhile, improve catalyst separation and recycling.

### BACKGROUND OF THE INVENTION

1. Field of the Invention

The invention generally relates to immobilized ionic liquids. More particularly, the invention relates to ionic liquids immobilized on a layered support material.

2. Description of the Relevant Art

Ionic liquids have attracted significant attention and have been extensively studied over the past decade. Because of their unique chemical and physical properties and the facile property tunability, ionic liquids not only have been used as alternatives to classical molecular solvents in a wide range of applications but also have led to many new applications, such as catalysis, electrolytes, lubricants, biomass processing, energetic materials, etc.

However, the use of ionic liquids has two major issues: cost and viscosity. One of the most promising approaches to solve these two problems is to immobilize ionic liquids on solid supports. In fact, immobilization of ionic liquids can also increase efficiency, facilitate recycling, and bring about new applications. For example, for ionic liquid catalyzed reactions, supported ionic liquids can bring a number of advantages, including facilitating catalyst separation, increasing catalysis efficiency, minimizing product contamination, and opening the possibility to use fixed-bed reactor systems. In addition, immobilization of ionic liquids can minimize the potential toxicity of ionic liquids, which has been largely ignored but has recently begun to draw attention. Thus far, porous silica and zeolite have been the main solid supports of choice.

### SUMMARY OF THE INVENTION

In one embodiment, an immobilized ionic liquid is formed using a layered material. In an embodiments, a composition includes a layered material; and an ionic liquid at least partially intercalated into the layered material.

In an embodiment, the layered material is α-zirconium phosphate [(Zr(HPO₄)₂.H₂O, α-ZrP]layered material. In another embodiment, the layered material is a smectite clay such as montmorillonite or laponite. In an embodiment, the ionic liquid is an imidazolium salt. In a specific embodiment, the layered material is α-zirconium phosphate and the ionic liquid is 1-butyl-3-methylimidazolium chloride (BMIMCl). The composition may include at least about 20% or at least about 40% of the ionic liquid intercalated into the layered material.

In an embodiment, a method of making a supported ionic liquid includes contacting an ionic liquid with a layered material; and mechanically mixing the ionic liquid with the layered material such that at least a portion of the ionic liquid is intercalated into the layered material. In some embodiments, mechanically mixing the ionic liquid with the layered material includes using a mechanical milling device such as ball miller. In an embodiment, mechanically mixing the ionic liquid with the layered material comprises using a mortar grinder. Mechanically mixing the ionic liquid with the layered material may be performed in the substantial absence of a solvent.

In an embodiment, a method of forming carbonate compounds includes coupling carbon dioxide to an epoxide in the presence of a catalyst, wherein the catalyst includes a layered material and an ionic liquid at least partially intercalated into the layered material. In some embodiments, the epoxide is propylene oxide, and the carbonate produced is propylene carbonate.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts X-ray diffraction (XRD) patterns of two α-ZrP samples prepared by different method [ZrP(3M-RF) and ZrP(6M-HT)]. The insets show the SEM images of ZrP(3M-RF) and ZrP(6M-HT);

FIG. 2 depicts XRD patterns of ZrP(3M-RF)/BMIMCl intercalation compounds with various BMIMCl loadings;

FIG. 3 depicts XRD patterns of ZrP(6M-HT)/BMIMCl intercalation compounds with various BMIMCl loadings; and

FIG. 4 depicts thermogravimetric (TGA) thermograms of pristine ZrP(3M-RF), BMIMCl, and ZrP(3M-RF)/BMIMCl intercalation compounds with various BMIMCl loadings.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

In one embodiment, an immobilized ionic liquid is formed by associating an ionic liquid material with a support. In some embodiments, the support is a layered material. An immobilized ionic liquid composition includes a layered material; and an ionic liquid at least partially intercalated into the layered material.

Layered materials are materials that are composed of stacked layers. Generally, layered materials expand in the presence of water and/or organic/inorganic compounds by allowing intercalation of the guest species between the stacked layers, causing the layers to expand.

In addition to immobilize ionic liquids on the surface of layered materials, ionic liquids can also be immobilized within the galleries of layered materials. In this way, the immobilized ionic liquids can be better protected, and the release of ionic liquids from the interlayer space might also be controlled, which would be very beneficial for certain applications. In addition, such a layered structure might be ideal for some specific applications, such as electrolytes.

One class of layered materials includes smectite clays. Examples of smectite clays include, but are not limited to, montmorillonite, bentonite, beidellite, nontronite, saponite, hectorite, stevensite and sauconite. Also encompassed are smectite clays prepared synthetically, e.g. by hydrothermal processes as disclosed in U.S. Pat. Nos. 3,252,757; 3,586,468; 3,666,407; 3,671,190; 3,844,978; 3,844,979; 3,852,405; and 3,855,147.

Other layered materials include, but are not limited to: Phosphates of titanium, zirconium, cerium, thorium, germanium, tin, lead, and vanadium(IV) (e.g., α-ZrP); Titanates having the composition M₂Ti₂O₅; M₂Ti₃O₇; M₂Ti₄O₉; M₂Ti₅O₁₁; M₂Ti₇O₁₅; etc where M is a univalent cation such as Li⁺, Na⁺, K⁺, NH₄⁺; Titanium niobates such as MTiNbO₅; M₃Ti₅NbO₁₄; MTi₂NbO₇; etc. where M is a univalent cation such as Li⁺, Na⁺, K⁺, NH₄⁺, and the like; Antimonates such as KSbO₃.H₂O and H₃Sb₃P₂O₁₄.H₂O and comparable niobates; Manganates such as NaMnO₂;. Na_{0.7}MnO₂; and Na_{0.7}MnO_{2.25}; Layered silicates such as magadiite H₂Si₁₄O₂₄; and Other layered oxides such as V₂O₅, MoO₃, WO₃, and UO₃ and their derivatives such as Ag₆Mo₁₀O₃₃.

Other layered materials also include: graphite, black-phosphorus, metal chalcogenides, metal oxides, metal oxy-halides, metal halides, hydrous metal oxides, layered double hydroxides, coordination compounds, silicides.

Layered α-ZrP may be used as a support to immobilize ionic liquids because of its high ion-exchange capacity, highly ordered structure, ease of synthesis, and ease of crystallinity and size control.

Layered materials may be used as a support to immobilize ionic liquids. As used herein the term ionic liquids refers to salts that exist in the liquid state at temperatures below about 100° C. Examples of salts that are ionic liquids include ammonium salts, choline salts, dibutyl phosphate, imidazolium salts, phosphonium salts, pyridinium salts, pyrazolium salts, pyrrolidinium salts, and sulfonium salts. Counter anions for these salts include acetate, aminoacetate; benzoate, bis(trifluoromethylsulfonyl)imide, dibutyl phosphate, dicyanamide, halides (fluorine, chorine, bromine, iodine, tribromide, triiodine), heptadecafluorooctanesulfonate, hexafluoroantimonate, hexafluorophosphate, hydrogen carbonate, hydrogen sulfate, hydroxide, lactate, methanesulfonate, 2-(2-methoxyethoxy)ethyl sulfate, methyl carbonate, methyl sulfate, nitrite, nonafluorobutanesulfonate, octyl sulfate; succinimide, tetrachloroaluminate, tetrafluoroborate, thiocyanate, thiophenolate, thiosalicylate, tosylate, trifluoroacetate and trifluoromethanesulfonate.

Examples of ammonium ionic liquids include, but are not limited to: Benzyldimethyltetradecylammonium chloride; Benzyltrimethylammonium tribromide purum; Butyltrimethylammonium bis(trifluoromethylsulfonyl)imide; Diethylmethyl(2-methoxyethyl)ammonium bis(trifluoromethylsulfonyl)imide; Ethyldimethylpropylammonium bis(trifluoromethylsulfonyl)imide; 2-Hydroxyethyl-trimethylammonium L-(+)-lactate; Methyltrioctadecylammonium bromide; Methyl-trioctylammonium bis(trifluoromethylsulfonyl)imide; Methyl-trioctylammonium bis(trifluoromethylsulfonyl)imide; Methyltrioctylammonium hydrogen sulfate; Methyltrioctylammonium thiosalicylate; Tetrabutylammonium benzoate; Tetrabutylammonium bis-trifluoromethanesulfonimidate; Tetrabutylammonium heptadecafluorooctanesulfonate; Tetrabutylammonium hydroxide; Tetrabutylammonium methanesulfonate; Tetrabutylammonium nitrite; Tetrabutylammonium nonafluorobutanesulfonate; Tetrabutylammonium succinimide; Tetrabutylammonium thiophenolate; Tetrabutylammonium tribromide; Tetrabutylammonium triiodide; Tetradodecylammonium bromide; Tetradodecylammonium chloride; Tetraethylammonium trifluoromethanesulfonate; Tetraheptylammonium bromide; Tetraheptylammonium chloride; Tetrahexadecylammonium bromide; Tetrahexylammonium bromide; Tetrahexylammonium hydrogensulfate; Tetrahexylammonium iodide; Tetrahexylammonium tetrafluoroborate; Tetrakis(decyl)ammonium bromide; Tetramethylammonium hydroxide pentahydrate; Tetraoctylammonium bromide; Tetraoctylammonium chloride; Tetrapentylammonium bromide; Tributylmethylammonium chloride; Tributylmethylammonium dibutyl phosphate; Tributylmethylammonium methyl carbonate; Tributylmethylammonium methyl sulfate; Triethylmethylammonium dibutyl phosphate; Triethylmethylammonium methyl carbonate; and Tris(2-hydroxyethyl)methylammonium methylsulfate.

Examples of imidazolium ionic liquids include, but are not limited to: 1-Allyl-3-methylimidazolium bis(trifluoromethylsulfonyl)imide; 1-Allyl-3-methylimidazolium bromide; 1-Allyl-3-methylimidazolium chloride; 1-Allyl-3-methylimidazolium dicyanamide; 1-Allyl-3-methylimidazolium iodide; 1-Benzyl-3-methylimidazolium chloride; 1-Benzyl-3-methylimidazolium hexafluorophosphate; 1-Benzyl-3-methylimidazolium tetrafluoroborate; 1,3-Bis(cyanomethyl)imidazolium bis(trifluoromethylsulfonyl)imide; 1,3-Bis(cyanomethyl)imidazolium chloride; 1,3-Bis(3-cyanopropyl)imidazolium bis(trifluoromethylsulfonyl)imide; 1,3-Bis(3-cyanopropyl)imidazolium chloride; 1-Butyl-2,3-dimethylimidazolium chloride; 1-Butyl-2,3-dimethylimidazolium hexafluorophosphate; 1-Butyl-2,3-dimethylimidazolium tetrafluoroborate; 4-(3-Butyl-1-imidazolio)-1-butanesulfonate; 4-(3-Butyl-1-imidazolio)-1-butanesulfonic acid triflate; 1-Butyl-3 -methylimidazolium acetate; 1-Butyl-3-methylimidazolium bis(trifluoromethylsulfonyl)imide; 1-Butyl-3-methylimidazolium bis(trifluoromethylsulfonyl)imide; 1-Butyl-3-methylimidazolium bromide; 1-Butyl-3-methylimidazolium chloride; 1-Butyl-3-methylimidazolium dibutyl phosphate; 1-Butyl-3-methylimidazolium dicyanamide; 1-Butyl-3-methylimidazolium hexafluoroantimonate; 1-Butyl-3-methylimidazolium hexafluorophosphate; 1-Butyl-3-methylimidazolium hydrogen carbonate; 1-Butyl-3-methylimidazolium hydrogen sulfate; 1-Butyl-3-methylimidazolium iodide; 1-Butyl-3-methylimidazolium methanesulfonate; 1-Butyl-3-methylimidazolium 2-(2-methoxyethoxy)ethyl sulfate; 1-Butyl-3-methyl-imidazolium methyl carbonate; 1-Butyl-3-methylimidazolium methyl sulfate; 1-Butyl-3-methylimidazolium nitrate; 1-Butyl-3-methylimidazolium octyl sulfate; 1-Butyl-3-methylimidazolium tetrachloroaluminate; 1-Butyl-3-methylimidazolium tetrafluoroborate; 1-Butyl-3-methylimidazolium thiocyanate; 1-Butyl-3-methylimidazolium tosylate; 1-Butyl-3-methylimidazolium trifluoroacetate; 1-Butyl-3-methylimidazolium trifluoromethanesulfonate; 1-Butyl-1-(3,3,4,4,5,5,6,6,7,7,8,8,8-tridecafluorooctyl)imidazolium hexafluorophosphate; 1-(3-Cyanopropyl)-3-methylimidazolium bis(trifluoromethylsulfonyl)amide; 1-(3-Cyanopropyl)-3-methylimidazolium chloride; 1-(3-Cyanopropyl)-3-methylimidazolium dicyanamide; 1-Decyl-3-methylimidazolium chloride; 1-Decyl-3-methylimidazolium tetrafluoroborate; 1,3-Diethoxyimidazolium bis(trifluoromethylsulfonyl)imide; 1,3-Diethoxyimidazolium hexafluorophosphate; 1,3-Dihydroxyimidazolium bis(trifluoromethylsulfonyl)imide; 1,3-Dihydroxy-2-methylimidazolium bis(trifluoromethylsulfonyl)imide; 1,3-Dimethoxyimidazolium bis(trifluoromethyl-sulfonyl)imide; 1,3-Dimethoxyimidazolium hexafluorophosphate; 1,3-Dimethoxy-2-methylimidazolium bis(trifluoromethylsulfonyl)imide; 1,3-Dimethoxy-2-methylimidazolium hexafluorophosphate; 1,3-Dimethylimidazolium dimethyl phosphate; 1,3-Dimethylimidazolium hydrogen carbonate; 1,3-Dimethylimidazolium methanesulfonate; 1,3-Dimethylimidazolium methyl sulfate; 1,2-Dimethyl-3-propylimidazolium bis(trifluoromethylsulfonyl)imide; 1,2-Dimethyl-3-propylimidazolium tris(trifluoromethylsulfonyl)methide; 1-Dodecyl-3-methylimidazolium iodide; 1-Ethyl-2,3-dimethylimidazolium tetrafluoroborate; 1-Ethyl-2,3-dimethylimidazolium chloride; 1-Ethyl-2,3-dimethylimidazolium ethyl sulfate; 1-Ethyl-2,3-dimethylimidazolium hexafluorophosphate; 1-Ethyl-2,3-dimethylimidazolium methyl carbonate; 1-Ethyl-2,3-dimethylimidazolium trifluoromethanesulfonate; 1-Ethyl-3-methylimidazolium acetate; 1-Ethyl-3-methylimidazolium aminoacetate; 1-Ethyl-3-methylimidazolium (S)-2-aminopropionate; 1-Ethyl-3-methylimidazolium bis(pentafluoroethylsulfonyl)imide; 1-Ethyl-3-methylimidazolium bis(trifluoromethylsulfonyl)imide; 1-Ethyl-3-methylimidazolium bromide; 1-Ethyl-3-methylimidazolium chloride; 1-Ethyl-3-methylimidazolium dibutyl phosphate; 1-Ethyl-3-methylimidazolium dicyanamide; 1-Ethyl-3-methylimidazolium diethyl phosphate; 1-Ethyl-3-methylimidazolium dimethyl phosphate; 1-Ethyl-3-methylimidazolium ethyl sulfate; 1-Ethyl-3-methylimidazolium hexafluorophosphate; 1-Ethyl-3-methylimidazolium hydrogen carbonate; 1-Ethyl-3-methylimidazolium hydrogen sulfate; 1-Ethyl-3-methylimidazolium iodide; 1-Ethyl-3-methylimidazolium L-(+)-lactate; 1-Ethyl-3-methylimidazolium methanesulfonate; 1-Ethyl-3-methyl-imidazolium methyl carbonate; 1-Ethyl-3-methylimidazolium methyl sulfate; 1-Ethyl-3-methylimidazolium nitrate; 1-Ethyl-3-methylimidazolium tetrachloroaluminate; 1-Ethyl-3-methylimidazolium tetrachloroaluminate; 1-Ethyl-3-methylimidazolium tetrafluoroborate; 1-Ethyl-3-methylimidazolium 1,1,2,2-tetrafluoroethanesulfonate; 1-Ethyl-3-methylimidazolium thiocyanate; 1-Ethyl-3-methylimidazolium tosylate; 1-Ethyl-3-methylimidazolium trifluoromethanesulfonate; 1-Hexyl-3-methylimidazolium bis(trifluormethylsulfonyl)imide; 1-Hexyl-3-methylimidazolium chloride; 1-Hexyl-3-methylimidazolium chloride; 1-Hexyl-3-methylimidazolium hexafluorophosphate; 1-Hexyl-3-methylimidazolium iodide; 1-Hexyl-3-methylimidazolium tetrafluoroborate; 1-Hexyl-3-methylimidazolium trifluoromethansulfonate; 1-(2-Hydroxyethyl)-3-methylimidazolium dicyanamide; 1-Methylimidazolium chloride; 1-Methylimidazolium hydrogen sulfate; 1-Methyl-3-octylimidazolium chloride; 1-Methyl-3-octylimidazolium hexafluorophosphate; 1-Methyl-3-octylimidazolium tetrafluoroborate; 1-Methyl-3-octylimidazolium trifluoromethanesulfonate; 1-Methyl-3-propylimidazolium iodide; 1-Methyl-3-propylimidazolium methyl carbonate; 1-Methyl-3-(3,3,4,4,5,5,6,6,7,7,8,8,8-tridecafluorooctyl)imidazolium hexafluorophosphate; 1-Methyl-3-vinylimidazolium methyl carbonate; 1,2,3-Trimethylimidazolium methyl sulfate; and 1,2,3-Trimethylimidazolium trifluoromethanesulfonate.

Examples of phosphonium ionic liquids include, but are not limited to: Tetrabutylphosphonium methanesulfonate; Tetrabutylphosphonium tetrafluoroborate; Tetrabutylphosphonium p-toluenesulfonate; Tributylmethylphosphonium dibutyl phosphate; Tributylmethylphosphonium methyl carbonate; Tributylmethylphosphonium methyl sulfate; Triethylmethylphosphonium dibutyl phosphate; Trihexyltetradecylphosphonium bis(trifluoromethylsulfonyl)amide; Trihexyltetradecylphosphonium bis(2,4,4-trimethylpentyl)phosphinate; Trihexyltetradecylphosphonium bromide; Trihexyltetradecylphosphonium chloride; Trihexyltetradecylphosphonium decanoate; Trihexyltetradecylphosphonium dicyanamide; 3-(Triphenylphosphonio)propane-1-sulfonate; and 3-(Triphenylphosphonio)propane-1-sulfonic acid tosylate.

Examples of pyridinium ionic liquids include, but are not limited to: 1-Butyl-3-methylpyridinium bis(trifluormethylsulfonyl)imide; 1-Butyl-4-methylpyridinium bromide; 1-Butyl-4-methylpyridinium chloride; 1-Butyl-4-methylpyridinium hexafluorophosphate; 1-Butyl-4-methylpyridinium iodide; 1-Butyl-4-methylpyridinium tetrafluoroborate; 1-Butylpyridinium bromide; 1-(3-Cyanopropyl)pyridinium bis(trifluoromethylsulfonyl)imide; 1-(3-Cyanopropyl)pyridinium chloride; 1-Ethylpyridinium tetrafluoroborate; and 3-Methyl-1-propylpyridinium bis(trifluormethylsulfonyl)imide.

Examples of pyrrolidinium ionic liquids include, but are not limited to: 1-Butyl-1-methylpyrrolidinium bis(trifluoromethylsulfonyl)imide; 1-Butyl-1-methylpyrrolidinium bromide; 1-Butyl-1-methylpyrrolidinium chloride; 1-Butyl-1-methylpyrrolidinium dicyanamide; 1-Butyl-1-methylpyrrolidinium hexafluorophosphate; 1-Butyl-1-methylpyrrolidinium iodide; 1-Butyl-1-methylpyrrolidinium methyl carbonate; 1-Butyl-1-methylpyrrolidinium tetrafluoroborate; 1-Butyl-1-methylpyrrolidinium trifluoromethanesulfonate; 1-Ethyl-1-methylpyrrolidinium bis(trifluoromethylsulfonyl)imide; 1-Ethyl-1-methylpyrrolidinium bromide; 1-Ethyl-1-methylpyrrolidinium hexafluorophosphate; and 1-Ethyl-1-methylpyrrolidinium tetrafluoroborate.

Other examples of ionic liquids that may be immobilized in a layered support include, but are not limited to: 1,2,4-Trimethylpyrazolium methylsulfate; Triethylsulfonium bis(trifluoromethylsulfonyl)imide; Bis(pentafluoroethylsulfonyl)imide; 1-Butyl-1-methylpiperidinium tetrafluoroborate; 1-Butyl-1-methylpiperidinium bis(trifluoromethylsulfonyl)imide; 1-Butyl-1-methylpiperidinium hexafluorophosphate; 4-Ethyl-4-methylmorpholinium methyl carbonate; 1-Ethyl-1-methylpiperidinium methyl carbonate; and Cholin acetate.

Direct intercalation of ionic liquids using an aqueous solution has been attempted, but generally is not successful. For example, ionic liquids were not able to be introduced into the gallery of α-zirconium phosphate until the α-zirconium phosphate was pre-intercalated by butylamine. Although the two-step intercalation might be acceptable for some applications, the pre-intercalated amine is highly undesirable or unacceptable for many applications, such as catalysis. The unsuccessful intercalation of bulky ionic liquids into layered compounds in solution systems is mainly owing to the dimensional mismatch. When the dimension of ionic liquids is larger than the interlayer gap of layered compounds, simple stirring or ultrasonication is usually not sufficient to force the guest ionic liquids into the host layered compounds. To solve the problem, it was discovered that ionic liquids may be incorporated into layered materials via a mechanochemical approach. Unlike a regular solution intercalation route, this route can be proceeded under ambient or high-temperature conditions via adsorption, a displacement or functional reaction, with prime benefits of not requiring solvent, higher production yield, and short reaction time (as short as a few minutes).

Ionic liquids may be immobilized on a support by contacting an ionic liquid with a layered material. The mixture of ionic liquid and the layered material are mechanically mixed such that at least a portion of the ionic liquid is intercalated into the layered material. Mechanical mixing may be accomplished using a number of known mechanical mixing devices. Examples of techniques that may be used for performing mechanical mixing include, but are not limited to, impact milling, attrition milling, knife milling, ball-milling, and direct-pressure milling.

Impact milling occurs when a hard object that applies a blunt force across a wide area hits a particle to fracture it. This milling action may be produced by a rotating assembly that uses blunt or hammer-type blades. Another type of impact mill is a jet mill. A jet mill uses compressed gas to accelerate the particles, causing them to impact against each other in the process chamber. Impact mills can reduce both fine powders and large chunks of friable material down to average particle sizes of 50 μm with mechanical impact mills, and less than 10 μm with jet mills. Mechanical impact mill types include hammer mills, pin mills, cage mills, universal mills, turbo mills and mortar grinders.

In attrition milling, nondegradable grinding media continuously contacts the material, systematically grinding its edges down. This milling action is typically produced by a horizontal rotating vessel filled with grinding media and tends to create free-flowing, spherical particles. Attrition mills can reduce materials down to an average particle size of less than 1 μm. One type of attrition mill is the media mill (also called a ball mill).

In knife milling, a sharp blade applies high, head-on shear force to a large particle, cutting it to a predetermined size to create smaller particles and minimize fines. This milling action is produced by a rotating assembly that uses sharp knives or blades to cut the particles. Mill types include knife cutters, dicing mills, and guillotine mills.

In ball milling, the ball mills rotate around a horizontal axis, partially filled with the material to be ground plus the grinding medium. Different materials are used as media, including ceramic balls, flint pebbles and stainless steel balls. An internal cascading effect reduces the material to a fine powder.

Direct-pressure milling occurs when a particle is crushed or pinched between two hardened surfaces. Two rotating bars or one rotating bar and a stationary plate generally produce this milling action. Direct-pressure mills typically reduce friable materials down to 800 to 1,000 μm. Types include roll mills, cracking mills, and oscillator mills.

In some embodiments, mechanical mixing devices provide sufficient energy to allow at least partial intercalation of the ionic liquid material into the layers of the layered materials. An advantage of such a process is that the intercalation may take place in the absence of a substantial amount of solvent. The lack of a solvent makes this process less expensive and more environmentally acceptable.

The amount of the ionic liquid intercalated into the layered material may be controlled based on the ratio of ionic liquid to layered material. In some embodiments, the ionic liquid may be incorporated at about 1% to about 80% by weight; from about 5% to about 75% by weight; from about 10% to about 70% by weight; or from about 15% to about 60% by weight. In some embodiments, additional ionic liquid may be adsorbed onto the layered material when the intercalation capacity of the layered material is reached.

Immobilized ionic liquids that are at least partially intercalated in a layered material may be used in any applications that the ionic liquid is usually applied. Because of their unique chemical and physical properties and the facile property tunability, supported ionic liquids may be used as alternatives to classical molecular solvents in a wide range of applications. Immobilized ionic liquids may also be used in applications such as catalysis, electrolytes, lubricants, biomass processing, and as energetic materials.

Due to the unique properties of ionic liquids in their liquid state, one use is as catalysts for many different types of reactions. In one embodiment, immobilized ionic liquids may be used as a catalyst for the formation of carbonates from carbon dioxide and epoxides. The conversion of CO₂ to valuable chemicals has long been a challenge, and it has recently attracted particular interest owing to the climate issues related to CO₂. One potential approach is the coupling reaction of CO₂ and epoxides to synthesize cyclic carbonates, which has wide applications. Many catalytic systems have been developed for the coupling reaction, including ionic liquids, most of which suffer from serious issues, such as low catalytic activity and/or selectivity, low stability, requiring cosolvent, requiring high pressure/temperature, etc. Although ionic liquids have been demonstrated to be effective in the chemical fixation of carbon dioxide, it is much more desirable to use immobilized ionic liquids, as discussed above.

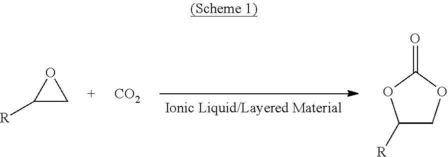

Scheme 1 depicts the general reaction, where R is hydrogen or a C₁-C₆ alkyl. Combining CO₂ gas with the epoxides in the presence of an immobilized ionic liquid produces the corresponding carbonates in good yield. The reaction is run at temperatures at or above the melting point of the ionic liquid. The use of an immobilized ionic liquid allows the reaction to be run in the absence of a solvent, making the process efficient and cost effective, when compared with solvent based operations.

While the above description describes the immobilization of ionic liquids by layered material, it should be understood that other catalytic materials may also be immobilized onto a layered material using mechanochemical processing. The use of immobilized catalysts may be a particularly useful for reactions that use heterogeneous catalysts. The layered material immobilized catalysis may be used for any reaction that would use the catalyst in an non-immobillized state, or immobilized on a different kind of substrate. For example, catalysts immobilized on a layered material may be used for reactions such as the water-gas shift reaction, methanol production and ammonia synthesis. Typical catalysts used for these reactions can be immobilized on a layered support by use of mechanochemical processing.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Materials

Zirconyl chloride octahydrate (ZrOCl₂.8H₂O, 98%, Aldrich), phosphoric acid (85%, Aldrich), and BMIMCl (Aldrich) were used as received. Propylene oxide with a purity of 95.0% was pretreated by potassium hydroxide and refluxed over calcium hydride for 24 h. It was then distilled under dry nitrogen gas and stored over 4 Å molecular sieves prior to use.

α-ZrP platelets with different lateral dimensions were synthesized according to the procedures described in the paper by Sun et al. New J. Chem. 2007, 31, 39-43, which is incorporated herein by reference. Briefly, a sample of 10.0 g of ZrOCl₂.8H₂O was refluxed with 100.0 mL of 3.0 M H₃PO₄ at 100° C. for 24 h to synthesize ZrP (“3M-RF”). A sample of 6.0 g of ZrOCl₂.8H₂O was mixed with 60.0 mL of 6.0 M H₃PO₄ in a sealed Teflon-lined pressure vessel and reacted at 200° C. for 24 h to prepare ZrP (“6M-HT”). ZrP(3M-RF) and ZrP(6M-HT) were used as supports for ionic liquids.

The following mechanochemical reaction procedures were used to intercalate BMIMCl into ZrP to prepare a series of intercalation compounds. In an agate mortar, a predetermined amount of ZrP was first ground for 3 min. BMIMCl was then added to the mortar and ground with ZrP for 10 min to generate the intercalation compound. Various amounts of BMIMCl were reacted with two ZrP hosts, which were formulated based on the molar ratio of available cations in the guest BMIMCl to the total cation-exchange capacity of the host. For example, ZrP(3M-RF)-50 refers to a sample formulated with an amount of BMIMCl that counts 50% of the total exchangeable cations in ZrP(3M-RF).

X-ray diffraction (XRD) patterns were recorded on a Bruker D8 diffractometer with Bragg-Brentano θ-2θ geometry (20 kV and 5 mA), using a graphite monochromator with Cu KR radiation. The thermal stability of the intercalation compounds was characterized by a thermogravimetric analyzer (TGA, TA Instruments model Q50) under an air atmosphere (40 mL/min) at a heating rate of 10° C./min.

The catalysis application of the immobilized BMIMCl was evaluated through a coupling reaction of carbon dioxide (99.99%) and PO in a 100 mL stainless steel autoclave equipped with a magnetic stirrer. For a typical reaction process, the immobilized BMIMCl and PO were charged into the reactor, which was pressurized with carbon dioxide at 1.5 MPa and reacted at 110° C. for 10 h. The reactor was then cooled to room temperature, and the resulting mixture was filtered. The unreacted PO was separated by the distillation of the filtrate under vacuum, and the product propylene carbonate was collected.

### Results and Discussion

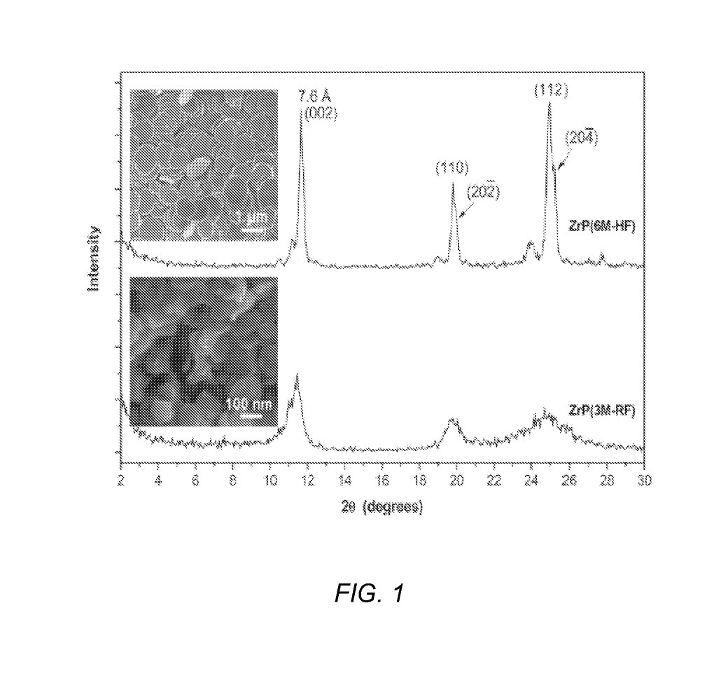

The unsuccessful direct intercalation of BMIMCl into ZrP in aqueous solution indicated that stirring and ultrasonication cannot supply sufficient energy to force the ILs into the gallery of ZrP in the solution state. To provide additional energy to help ILs overcome the intercalation energy barrier, a mechanochemical reaction approach was used. The two ZrP hosts synthesized for this project represent two model platelets with different lateral dimensions and levels of crystallinity, and thus different intercalation barriers, for comparison. XRD patterns and SEM images of the two ZrP samples are shown in FIG. 1. The patterns confirm the formation of ZrP. The two samples exhibit significantly different levels of crystallinity, as evidenced by different peak widths and signal-to-noise ratios, but both have an average interlayer distance of 7.6 Å. Furthermore, SEM images clearly show that both of the two ZrP samples exhibit a platelike structure, with a lateral dimension of approximately 80-100 and 800-1000 nm for ZrP(3M-RF) and ZrP(6M-HT), respectively. The broadened peaks for ZrP(3M-RF) are mainly owing to its less ordered layer stacking Such a stacking disorder in ZrP(3M-RF), together with its relatively low lateral dimension, may lower the overall intercalation energy barrier compared to ZrP(6M-HT) and thus will be beneficial for the mechanochemical intercalation.

It was observed that, after initial grinding, the ZrP (both ZrP(3M-RF) and ZrP(6M-HT)) and BMIMCl mixture formed a wet paste. This is understandable because, under the grinding pressure, BMIMCl turns to a liquid phase. Upon mixing with ZrP powders, they form a paste. As grinding proceeds, the paste gradually became drier and drier. For the samples with a low concentration of BMIMCl, the final products turned out to be dry powders, whereas the ones containing a high concentration of BMIMCl turned out to be a highly viscous suspension. This phase transition indicated that, at the earlier stage, a simple powder/liquid mixture formed. As the reaction proceeded, BMIMCl was gradually intercalated into the gallery and resulted in dry powder samples. This phenomenon indicates that the mechanochemical reaction can effectively enable BMIMCl to be intercalated into layered ZrP. Multiple samples with different hosts and various formulation ratios were prepared and are summarized in Table 1.

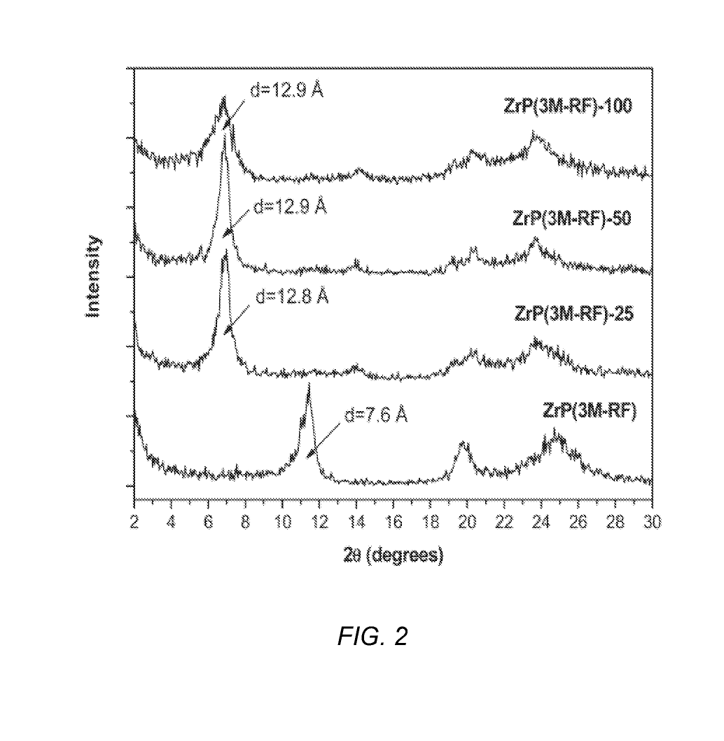

To verify the above hypothesis, the prepared samples were characterized by XRD. FIG. 2 presents the XRD patterns of ZrP(3M-RF)/BMIMCl intercalation compounds, which clearly show an increased interlayer distance after mechanochemical reaction. To be noted, even at a low formulation ratio of 25% (ZrP(3M-RF)-25), the peak at 7.6 Å corresponding to the pristine ZrP completely disappeared, while a new intensive peak located at 12.8 Å was observed on the pattern. This indicated that a new intercalation compound formed with no pristine ZrP left. At a formulation ratio of 100% (ZrP(3M-RF)-100), the reduced peak intensity indicates that a portion of the BMIMCl may not be intercalated into the gallery but instead may be adsorbed on the surface. This is also consistent with the paste appearance of the sample, as summarized in Table 1. The reason BMIM cations cannot be 100% intercalated into ZrP is believed to be owing to the high density of cation-exchange sites (hydroxyl groups) in ZrP, while the BMIM cation has a dimension larger than the distance between neighboring hydroxyl groups. The steric hindrance prevents 100% intercalation.

TABLE 1
Formulation and appearance of ZrP/BMIMCl intercalation compounds.

| | BMIM⁺/ | BMIMCl weight | Product |
|---|---|---|---|
| Sample | exchangeable cation | percentage (wt %) | appearance |
| ZrP(3M-RF)-25 | 0.25:1 | 22.5 | Powder |
| ZrP(3M-RF)-50 | 0.50:1 | 36.7 | Powder |
| ZrP(3M-RF)-100 | 1.00:1 | 53.7 | Paste |
| ZrP(6M-HT)-25 | 0.25:1 | 22.5 | Powder |
| ZrP(6M-HT)-50 | 0.50:1 | 36.7 | Powder |
| ZrP(6M-HT)-100 | 1.00:1 | 53.7 | Paste |

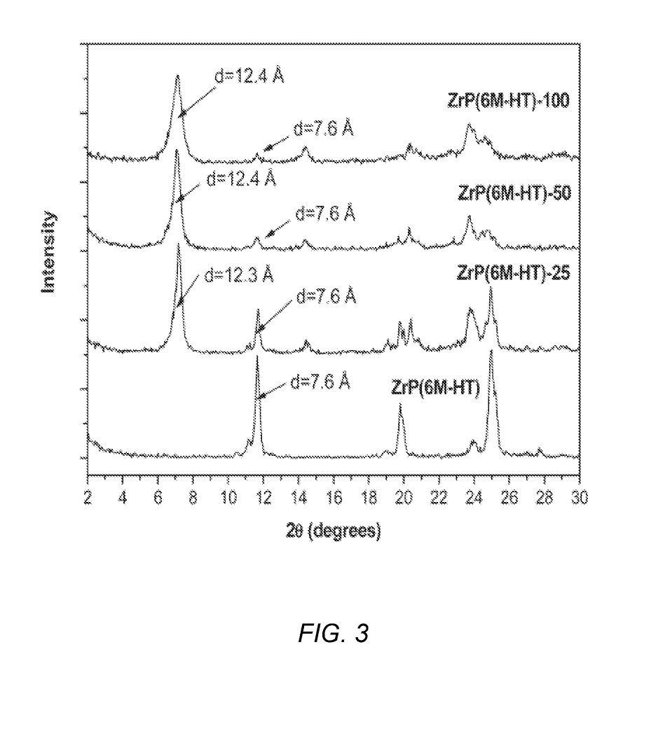

Similar mechanochemical intercalation results were achieved in ZrP(6M-HT), as shown in FIG. 3. However, when ZrP(6M-HT) was used as the host, the pristine ZrP phase cannot be completely removed. Although a higher BMIMCl loading led to a lower concentration of the pristine ZrP phase, even at a formulation ratio of 100%, a tiny amount of pristine ZrP still remained in the intercalation compound. This is believed to be mainly owing to the much larger lateral dimension of ZrP(6M-HT) (800-1000 nm) compared with ZrP(3M-HT) (80-100 nm). Because the intercalation of BMIM cations into the gallery progresses from the edge to the center, it is more difficult for BMIM cations to diffuse throughout the layer in larger platelets. Besides, the more ordered structure (higher crystallinity) of ZrP(6M-HT) compared with ZrP(3M-RF), and, therefore, higher Van Der Waals forces between the layers, should be another factor for the different intercalation behaviors.

It was noted that the interlayer distance for both ZrP(3M-RF) and ZrP(6M-HT)-based intercalation compounds only enhanced slightly with increasing concentration of BMIMCl in the formulation and saturated at ca. 12.9 and 12.4 Å, respectively. This is different from the solution intercalation results, which typically exhibit an enhanced interlayer distance with increasing intercalation ratio. The reason for such a different result is possibly owing to the nature of the reaction. During solution intercalation, ultrasonication is typically adopted to promote the insertion of guests into the host layers. The guest molecule vibration and localized heating generated by ultrasonication might effectively expand the host layer gradually, which allows more guest molecules to be intercalated into the gallery and then tilted. For mechanochemical intercalation, the mechanical force can only promote the insertion of guest molecules into the gaps of the layered compounds, but it was not able to effectively expand the interlayer distance. This is consistent with some earlier mechanochemical intercalation results, in which the interlayer distance of the montmorillonite/octadecylamine intercalation compound was independent of the concentration of octadecylamine. Heating the intercalated compounds prepared via the mechanochemical reaction did lead to further expansion of the interlayer distance, which supports the above conjecture.

The slight difference in the interlayer distance between the two series of samples is believed to be owing to the different levels of crystallinity between the two hosts. ZrP(3M-RF) is of lower crystallinity and less ordered and thus can be more easily intercalated, and thus the BMIM cations might also be slightly tilted. ZrP(6M-HT) is of much higher crystallinity and larger size, in which BMIM cations were almost perfectly parallel to the layers. Considering that the BMIM cation has a thickness of ca. 2.9 Å, the layer thickness of ZrP is ca. 6.3 Å, and ZrP(6M-HT)/BMIMCl intercalation compounds have an interlayer distance of ca. 12.4 Å, this indicates that a bilayer of BMIM cations stay virtually parallel to ZrP(6M-HT) layers, whereas a bilayer of BMIM cations with a small tilt angel should exist in ZrP(3M-RF) galleries.

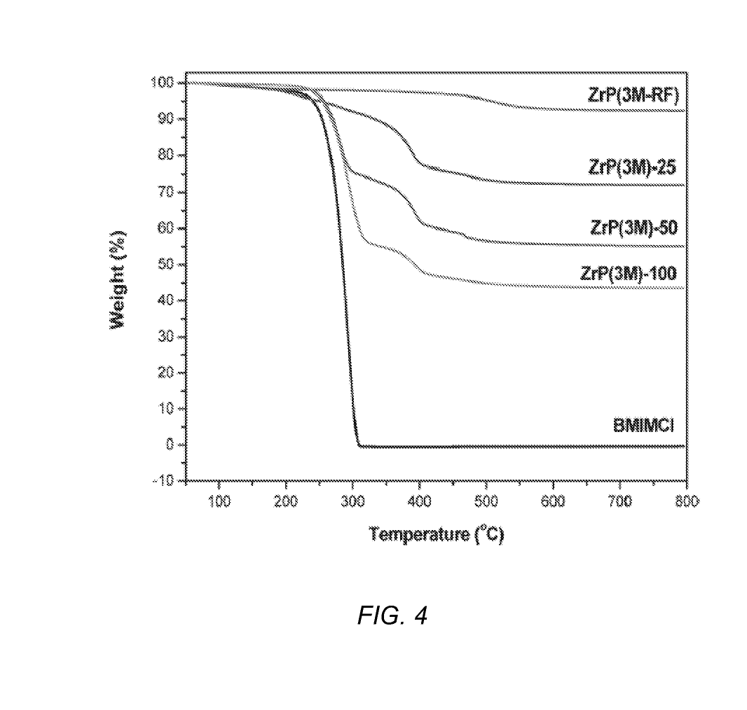

The thermal stability of the intercalation compounds was investigated by TGA. Prior to each test, the samples were isothermed at 90° C. for 30 min in an air flow to remove absorbed moisture, then cooled down to 50° C. to start the test. The TGA thermograms of ZrP(3M-RF)/BMIMCl intercalation compounds are shown in FIG. 4, with BMIMCl and neat ZrP(3M-RF) as the controls. The sharp weight loss of BMIMCl started from ca. 210° C., and it lost all the weight at ca. 315° C. ZrP exhibits a two-step degradation at ca. 100-170 and 450-580° C., corresponding to the removal of hydration water and condensation water, respectively. The ZrP(3M-RF)/BMIMCl intercalation compounds mainly exhibited three weight losses at ca. 220-320° C., 340-410° C., and 450-580° C. The first step of weight loss agrees well with the degradation of the BMIMCl control sample, but slightly delayed. This step of weight loss is owing to the degradation of BMIMCl adsorbed on ZrP surface. The increasing amount of adsorbed BMIMCl from sample ZrP(3M-RF)-25 to ZrP(3M-RF)-100 is also very consistent with the formulation. The second step of degradation can be attributed to the degradation of intercalated BMIMCl in the ZrP gallery. Because of the protection from the inorganic layers, and the electrostatic bonding with the layers, the degradation of this part of BMIMCl was delayed until ca. 340-410° C. The delayed degradation indirectly supports that BMIM cations were intercalated into the interlayer space via the mechanochemical reaction. The third step of degradation corresponding to the removal of condensation water in ZrP was not clearly seen in FIG. 4 but can be observed on the derivative curve (not shown). This is mainly because of the lowered weight concentration of ZrP in the intercalation compounds. Similar TGA results were obtained for ZrP(6M-HT)/BMIMCl intercalation compounds.

Formation of Carbonates from CO₂

The immobilized BMIMCl in ZrP was evaluated for catalysis applications using the following reaction.

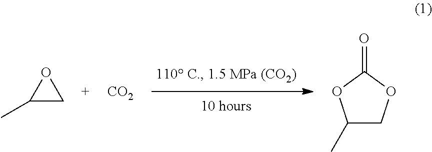

During the evaluation of the immobilized BMIMCl, no cosolvent or cocatalyst was used. The detailed reaction conditions and evaluation results are summarized in Table 2.

TABLE 2
Catalysis evaluation results for the formation of propylene carbonate via
the coupling reaction of CO₂ and propylene oxides.

| | | | | Propylene |
|---|---|---|---|---|
| | Amount of | Amount of | Pressure | carbonate Yield |
| Catalyst | catalyst (g) | PO (mL) | (MPa) | (%) |
| BMIMCl | 0.537 | 15.0 | 1.5 | 55.9 |
| ZrP(3M-RF)-100 | 1.000 | | | 46.1 |
| ZrP(6M-HT)-100 | 1.000 | | | 55.1 |

When 0.537 g of BMIMCl was used as the catalyst, the yield was 55.9%, whereas when 1.000 g of immobilized catalysts ZrP(3M-RF)-100 and ZrP(6M-HT)-100 (both containing 0.537 g of BMIMCl) was used, yields of 46.1 and 55.1% were achieved, respectively. The results showed that the immobilized BMIMCl can maintain a similar level of reactivity as free BMIMCl. ZrP(6M-HT)-100 exhibited a higher reactivity than ZrP(3M-RF)-100 during this catalysis evaluation, possibly owing to the more uniform intercalation and adsorption of BMIMCl on ZrP(6M-HT) layers.

Ionic liquids have been widely considered as green solvents and used in many green chemistry applications. The facile mechanochemical reaction approach can effectively immobilize BMIMCl in layered compounds within minutes without using any solvent. Meanwhile, the immobilized ionic liquids could perform more effectively and find promising applications, such as being used as catalysts for green chemical reactions, that is, the fixation of CO₂. The mechanochemical reaction approach can thus be considered as a “green” approach (no solvent, low energy consumption, etc.), which renders ionic liquids to be “greener” after immobilization.

Other chemicals, such as other ionic liquids and JeffamineM1000 amine (a solid-state polyoxyalkyleneamine fromHuntsmanCorporation) have also been successfully intercalated into ZrP via the mechanochemical reaction. Meanwhile, BMIMCl has been successfully immobilized in other layered compounds, such as montmorillonite. All these results show that the mechanochemical reaction can be adopted as a general approach to intercalate large molecules, which are difficult to be intercalated in solution state, into layered compounds.

### Conclusions

The mechanochemical reaction has been proved to be a facile and effective approach to immobilize ionic liquids in layered compounds, as evidenced by both the XRD and TGA characterizations. Without using any solvent and requiring only a few minutes of the single-step reaction, the mechanochemical reaction serves as a “green” approach to immobilize “green” ionic liquids to be “greener”, considering that the immobilized ionic liquids could perform more effectively and efficiently for practical applications (such as catalysis). In addition, the mechanochemical reaction conducted in the lab using a mortar and pestle can be easily scaled up in industry using tools such as a ball-miller. Thus, it is expected that the mechanochemical reaction can be easily adopted for industrial applications.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A composition comprising:
a layered material;
an ionic liquid at least partially intercalated into the layered material.

2. The composition of claim 1, wherein the layered material is α-ZrP layered material.

3. The composition of claim 1, wherein the layered material is montmorillonite.

4. The composition of claim 1, wherein the layered material is laponite.

5. The composition of claim 1, wherein the ionic liquid is an imidazolium salt.

6. The composition of claim 1, wherein the layered material is α-ZrP and the ionic liquid is BMIMCl.

7. The composition of claim 1, wherein the composition comprises at least 40% of the ionic liquid intercalated into the layered material.

8. A method of making a supported ionic liquid comprising:
contacting an ionic liquid with a layered material;
mechanically mixing the ionic liquid with the layered material such that at least a portion of the ionic liquid is intercalated into the layered material.

9. The method of claim 8, wherein mechanically mixing the ionic liquid with the layered material comprises using a mechanical milling device.

10. The method of claim 8, wherein mechanically mixing the ionic liquid with the layered material comprises using a mortar grinder.

11. The method of claim 8, wherein mechanically mixing the ionic liquid with the layered material is performed in the substantial absence of a solvent.

12-16. (canceled)

17. A composition comprising a layered material and an ionic liquid at least partially intercalated into the layered material made using the process of claim 8.

18. A method of forming carbonate compounds comprising coupling carbon monoxide to an epoxide in the presence of a catalyst, wherein the catalyst comprises a layered material and an ionic liquid at least partially intercalated into the layered material.

19. The method of claim 18, wherein the epoxide is propylene oxide, and wherein the carbonate produced is propylene carbonate.

20. The method of claim 18, wherein the layered material is α-ZrP.

21. The method of claim 18, wherein the layered material is montmorillonite.

22. The method of claim 18, wherein the layered material is laponite.

23. The method of claim 18, wherein the ionic liquid is an imidazolium salt.

24. The method of claim 18, wherein the layered material is α-zirconium phosphate and the ionic liquid is BMIMC1.

25. The method of claim 18, wherein the composition comprises at least 40% of the ionic liquid intercalated into the layered material.

26. (canceled)

27. (canceled)
